(19) United States
(12) Patent Application Publication
(10) Pub. No.: US 2019/0138687 A1
(43) Pub. Date: May 9, 2019

(54) IDENTIFICATION OF TARGET CONTENT IN METAGENOME SAMPLE

(71) Applicant: INTERNATIONAL BUSINESS MACHINES CORPORATION, Armonk, NY (US)

(72) Inventors: Niina S. HAIMINEN, Valhalla, NY (US); Laxmi P. PARIDA, Yorktown Heights, NY (US); Enrico SIRAGUSA, White Plains, NY (US); Filippo UTRO, Pleasantville, NY (US)

(21) Appl. No.: 15/802,874

(22) Filed: Nov. 3, 2017

(51) Int. Cl. G06F 19/24; G06F 19/22; G06F 17/18; C12Q 1/68

(52) U.S. Cl. CPC G06F 19/24; G06F 19/22; G06F 19/26; C12Q 1/689; G06F 17/18

(57) ABSTRACT

Embodiments include methods, systems, and computer program products for identifying content in a metagenomic sample. Aspects include receiving a plurality of metagenomic reads for a sample. Aspects also include comparing a portion of the plurality of metagenomic reads to a genomic database and identifying a plurality of associated nodes, wherein the genomic database includes a plurality of gene sequences having known taxonomies. Aspects also include generating a probabilistic score for each of the associated nodes per metagenomic read. Aspects also include generating an output including a plurality of identifications and a final probability score for each of the identifications based at least in part upon the probabilistic score for each of the associated nodes per metagenomic read.

### BACKGROUND

The present invention relates to metagenomic analysis, and more specifically, to methods, systems and computer program products for identification of target content in a metagenome sample.

Metagenome classification involves extraction and identification of genomic sequences from environmental or clinical samples. Environmental samples, such as soil samples, food samples, or biological tissue samples can contain desirable, neutral, or undesirable content that one seeks to identify. For instance, in some cases, target content is undesirable content such as viruses, bacteria, or other content that can interfere with the integrity of a sample. Metagenomic samples can include extremely large numbers of organisms and, consequently, generate a large set of genomic data. For example, it is estimated that the human body, which relies upon bacteria for modulation of digestive, endocrine, and immune functions, can contain up to 100 trillion organisms. In the past decade, advances in sequencing and screening technologies have increased the potential for determining the microbial composition of previously unknown samples and the identification of target content. It is desirable to determine the target content in a metagenome sample as precisely as possible.

### SUMMARY

In accordance with one or more embodiments, a computer-implemented method for identifying content in a metagenomic sample is provided. A non-limiting example of the method includes receiving, by a processor, a plurality of metagenomic reads for a sample. The method also includes comparing, by the processor, a portion of the plurality of metagenomic reads to a genomic database and identifying a plurality of associated nodes, wherein the genomic database includes a plurality of gene sequences having known taxonomies. The method also includes generating, by the processor, a probabilistic score for each of the associated nodes per metagenomic read. The method also includes generating an output including a plurality of identifications and a final probability score for each of the identifications based at least in part upon the probabilistic score for each of the associated nodes per metagenomic read.

In accordance with another embodiment, a computer program product for identifying content in a metagenomic sample is provided. The computer program product includes a computer readable storage medium readable by a processing circuit and storing program instructions for execution by the processing circuit for performing a method. A non-limiting example of the method includes receiving a plurality of metagenomic reads for a sample. The method also includes comparing a portion of the plurality of metagenomic reads to a genomic database and identifying a plurality of associated nodes, wherein the genomic database includes a plurality of gene sequences having known taxonomies. The method also includes generating a probabilistic score for each of the associated nodes per metagenomic read. The method also includes generating an output including a plurality of identifications and a final probability score for each of the identifications based at least in part upon the probabilistic score for each of the associated nodes per metagenomic read.

In accordance with a further embodiment, a processing system for identifying content in a metagenomic sample is provided. The processing system includes a processor in communication with one or more types of memory, the processor configured to perform a method. A non-limiting example of the method includes receiving a plurality of metagenomic reads for a sample. The method also includes comparing a portion of the plurality of metagenomic reads to a genomic database and identifying a plurality of associated nodes, wherein the genomic database includes a plurality of gene sequences having known taxonomies. The method also includes generating a probabilistic score for each of the associated nodes per metagenomic read. The method also includes generating an output including a plurality of identifications and a final probability score for each of the identifications based at least in part upon the probabilistic score for each of the associated nodes per metagenomic read.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The subject matter of the present invention is particularly pointed out and distinctly claimed in the claims at the conclusion of the specification. The foregoing and other features and advantages of the one or more embodiments described herein are apparent from the following detailed description taken in conjunction with the accompanying drawings in which:

FIG. 1 is block diagram illustrating one example of a processing system for practice of the teachings herein;

FIG. 2 is a flow diagram illustrating a method for characterizing metagenomic reads according to one or more embodiments of the present invention;

FIG. 3A is diagram illustrating aspects of a method for characterizing metagenomic reads according to one or more embodiments of the present invention;

FIG. 3B is diagram illustrating aspects of a method for characterizing metagenomic reads according to one or more embodiments of the present invention;

FIG. 3C is diagram illustrating aspects of a method for characterizing metagenomic reads according to one or more embodiments of the present invention;

FIG. 4 is a flow diagram illustrating a method for characterizing metagenomic reads according to one or more embodiments of the present invention;

FIG. 5 depicts a diagram illustrating an exemplary system for characterizing metagenomic reads according to one or more embodiments of the present invention;

FIG. 6 depicts a chart illustrating results of a method for characterizing metagenomic reads according to one or more embodiments of the present invention; and

FIG. 7 depicts a chart illustrating results of a method for characterizing metagenomic reads according to one or more embodiments of the present invention.

The diagrams depicted herein are illustrative. There can be many variations to the diagram or the operations described therein without departing from the spirit of the invention. For instance, the actions can be performed in a differing order or actions can be added, deleted, or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the described embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Metagenomics, the study of genomic species obtained directly from the environment or from human or other host organisms, is a desirable area of study that has increased in use over the past decade. Metagenomic sequencing can be computationally and experimentally challenging.

Metagenome sequencing can be performed in three stages. First, an environmental sample can be prepared. For instance, DNA from a sample can be isolated and then fragmented to obtain sequence fragments small enough for current sequencing techniques. Thereafter, sample preparation can include blunting the fragment ends and ligating adaptors to the DNA fragments, for instance, to enable substrate attachment in sequencing applications. Second, the prepared samples can be sequenced. Sequencing generally includes High Throughput Sequencing methods. Third, the sequence data can be analyzed with bioinformatics to identify and further analyze the genomic content of a sample.

Metagenomic sequencing provides mixtures of variable length nucleotide read originating from individual genomes. For many metagenomic samples, the species, genera, and even phyla present in the sample can be largely unknown at the time of sequencing. The nucleotide sequences generated from metagenomic samples, referred to as “reads,” must be mapped to their respective gene, or a species, genus, or other taxonomic entity (OTU, Operational Taxonomic Unit). A goal of sequencing can be to determine the contents of the composition, including in some cases the target content of the composition, as precisely as possible.

The large size of data sets generated through metagenomic sequencing poses analytical challenges. For instance, some conventional methods require the use of sequences of pre-defined length to perform an analysis, which are iteratively applied to a known nucleic acid sequence at overlapping positions. Although such iterative methods can yield accurate results, they can be computationally expensive and time consuming. Other methods can allow partial matches between a read and an indexed genomic sample, but such methods can include large numbers of false positives and false negatives that can be indistinguishable from true results.

Although some methods of identification of target content in metagenomic samples have been proposed, a conventional output can lack information needed to diagnose a result, such as statistical measures of probability or a means to resolve conflicting results, for instance due to sequencing errors. In some cases, a target content can be prohibitively large for computational analysis or laboratory-based techniques (such as polymerase chain reaction (PCR)), or conversely, too small for reliable detection and characterization.

Turning now to an overview of the aspects of the invention, one or more embodiments of the invention address the above-described shortcomings of the prior art by providing a method for identifying target content in a metagenome sample by determining, against a fixed threshold of tolerance, whether representatives from a specified set of target content are present. Embodiments of the invention provide read identification and probabilistic classification from a metagenomic sample against a database of multiple organisms. Some embodiments of the invention include building a metagenomic profile (e.g., species level abundance), comparing the profile to a pre-determined baseline profile, and identifying unexpected community variations above the threshold. Some embodiments of the invention utilize orientation of the read matches for characterization. Such embodiments can, for instance, limit acceptable matches to sequences having a complement or reverse complement to a known sequence in a defined direction or set of directions, such as inward from the 5′ and 3′ ends of a read and/or outward toward the 5′ and 3′ ends of a read, with respect to a reference sequence, to improve precision or accuracy of an identification and/or characterization.

The above-described aspects of the invention address the shortcomings of the prior art by obtaining a metagenomic sequencing sample and generating a probabilistic score for the reads indicating the presence and classification of target content based upon an indexed or annotated genomic database and a defined set of target content and associated tolerance. If distribution scores for target content exceed the threshold tolerances, in some embodiments an analysis can be terminated. In some embodiments of the invention, analysis can proceed until a desired accuracy is achieved.

Embodiments of the invention can provide a number advantages. For example, laboratory-only based methods, such as culture-based tests or rapid PCR tests, could be unworkable if a target content is too high. Moreover, some methods, such as culture-based methods can be impracticable in situations where time is limited, for instance as they can require prolonged waiting periods to achieve sufficient growth of relevant cultures. Combining sequencing with computational methods of embodiments of the invention, on the other hand, can provide additional information regarding antibiotic resistance and/or can potentially lead to the capture or near-capture of a whole genome sequence for precise and accurate characterization of target content. Embodiments of the invention can incur significant time and cost savings over conventional methods, for example by enabling the detection of all undesired content with performance of a single sequencing routine.

Embodiments of the invention can generate a distribution of probability scores that can be used to diagnose a result. For example, low probabilistic scores can indicate the presence of chimeric reads or organisms and, thus, can be filtered from results if desired. If different regions in a given read lead to conflicting identifications due to read errors, for instance, the more probable region can be determined and selected for read classification. Advantageously, embodiments of the invention can analyze and characterize any read lengths without the need to use overlapping k-mers of predefined length. Thus, embodiments of the invention desirably can reduce the required time and steps required for the analysis.

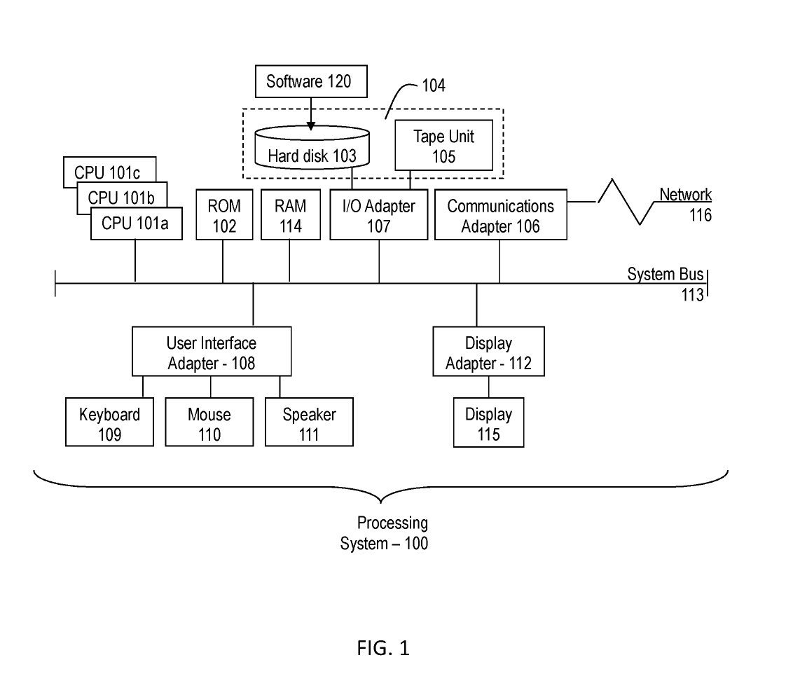

Turning now to a more detailed description of aspects of the present invention, FIG. 1 depicts an embodiment of a processing system 100 for implementing the teachings herein. In this embodiment, the system 100 has one or more central processing units (processors) 101a, 101b, 101c, etc. (collectively or generically referred to as processor(s) 101). In one embodiment, each processor 101 can include a reduced instruction set computer (RISC) microprocessor. Processors 101 are coupled to system memory 114 and various other components via a system bus 113. Read only memory (ROM) 102 is coupled to the system bus 113 and can include a basic input/output system (BIOS), which controls certain basic functions of system 100.

FIG. 1 further depicts an input/output (I/O) adapter 107 and a network adapter 106 coupled to the system bus 113. I/O adapter 107 can be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Software 120 for execution on the processing system 100 can be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which can include a graphics adapter to improve the performance of graphics intensive applications and a video controller. In one embodiment, adapters 107, 106, and 112 can be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which can include, for example, a Super I/O chip integrating multiple device adapters into a single integrated circuit.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

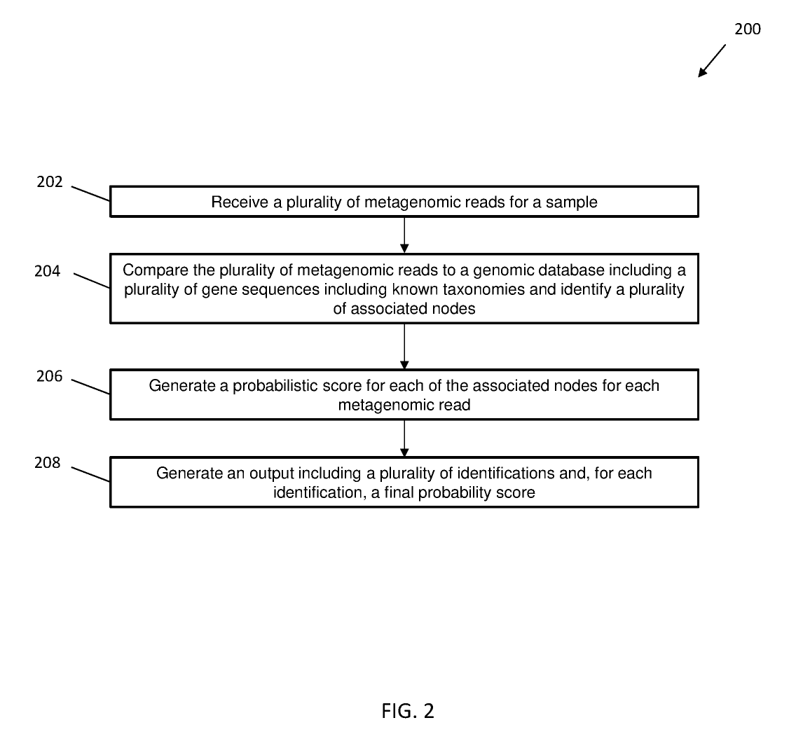

Turning now to a more detailed description of embodiments of the invention, FIG. 2 depicts a flow diagram illustrating an exemplary method 200 for characterizing a metagenomic sample according to one or more embodiments of the invention. The method 200 includes, as shown at block 202, receiving a plurality of metagenomic reads for a sample. The method 200 also includes, as shown at block 204, comparing the plurality of metagenomic reads to a genomic database including a plurality of gene sequences including known taxonomies, and identifying a plurality of associated nodes. The method 200 also includes, as shown at block 206, generating a probabilistic score for each of the associated nodes for each metagenomic read. The method 200 also includes, as shown at block 208 generating an output including a plurality of identifications and, for each identification, a final probability score.

In some embodiments, of the invention, the plurality of metagenomic reads are compared to the genomic database in multiple portions. For example, a first portion of a plurality of metagenomic reads can include full length reads and a second portion of the plurality of metagenomic reads can include a first set of fragments of the metagenomic reads. A set of fragments of the metagenomic reads can be a set of a fixed minimum length, a set that has been derived from a full length read by a fixed number of fragmentation rounds, or any other manner. Apportioning the reads and fragments of the reads can be determined by persons skilled in the art and can be performed, for example, according to desired computation speed or processing capabilities, and/or can depend upon the type of metagenomic sample and/or the desired application.

In some embodiments of the invention, a method includes repeating a comparison of the reads or read fragments to a genomic database. The repeated comparison can include the same set of reads or a different set or portion of reads or read fragments. The comparison can be repeated, for example, on iterative fragments of the reads until a match is found, or until a minimum fragment length, such as 15 bases or 20 bases, is reached. In some embodiments of the invention, in the case of pair-end reads, fragment orientation can be used to produce a concordant assignment.

Comparison of read fragments to a genomic database can include computationally comparing DNA sequences of the read to a known DNA sequence in an existing database, such as NCBI RefSeq. The genomic database can include one or more taxonomies associated with the known DNA sequences. In some embodiments of the invention, a full read, a fragmented read, and/or a reverse complement of a full read or a fragmented read, is compared to one or more sequences in the genomic database. Unless specifically indicated, a “read” is understood to include a full read, a read fragment (or seed), or a reverse complement of a read or read fragment. A full read or fragments of a read can be mapped to one or a plurality of nodes on a taxonomy. In some embodiments of the invention, a read is mapped to each of the bottom most nodes on a taxonomy tree.

An associated node includes a match between a read and a sequence or portion of a known sequence or portion of a known sequence for a taxonomy of the database.

Where nodes along a chain are in complete agreement with one another, and the lowest node represents the most specific node in a taxonomy tree, the node at the lowest level can provide the most specific available identification of a read or read fragment. In a taxonomy tree with multiple bottom-most nodes, the read can be compared to all of the bottom most nodes. In some embodiments of the invention, probabilities can be assigned to all of the bottom most nodes for each metagenomic read. Chains can be overlapping or non-overlapping. If chains in the taxonomic tree, including chains associated with the bottom-most nodes, do not overlap, the chains can be considered independent and probabilities can be calculated according to known methods. If chains in the taxonomic tree overlap, embodiments of the invention include computing probabilities based at least in part upon the dependencies of the nodes. In some embodiments of the invention, reads are mapped to the bottom most level of one or more taxonomies in a database. In some embodiments of the invention, reads are mapped to a fixed level of one or more taxonomies in a database.

For example, in some embodiments of the invention, a set of read fragments F for read r are defined and matched in database D such that associated nodes in taxonomy T are identified. Probabilistic scores for the set of nodes can be calculated by known methods. In some embodiments of the invention, read r is assigned to node T with the highest probabilistic score. Fragments can be obtained, for example, with heuristics such as iteratively splitting the read into two segments until one or more matches are identified in the database, until a minimum fragment length is reached, or until the analysis is complete or stopped by a user.

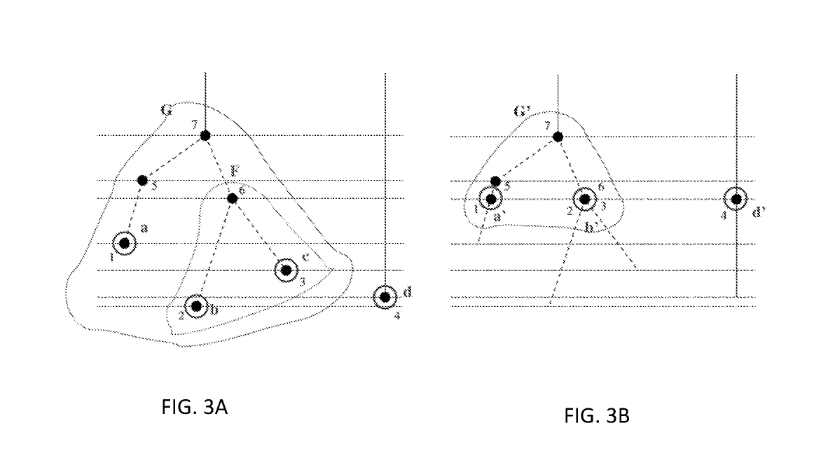

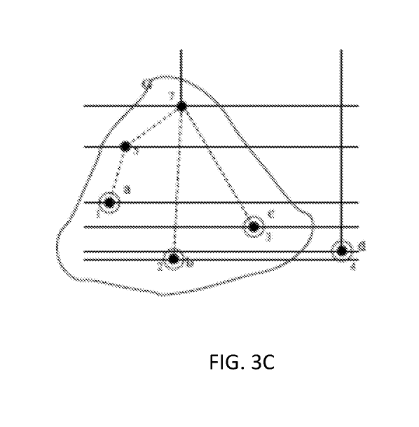

FIGS. 3A, 3B, and 3C depict aspects of exemplary probability determinations for associated nodes according to embodiments of the invention. FIGS. 3A-3B illustrate scoring of seven fragments of a single read on the taxonomy depicted as a dashed line tree (or forest), wherein the FIG. 3A illustrates scoring at the bottom-most level and FIG. 3B illustrates scoring at a fixed taxonomic level. The horizontal dotted lines in FIGS. 3A-3C correspond to different levels in the taxonomy. Read fragments 1-7 are mapped to the taxonomy at nodes indicated as black circles along the taxonomy trees. In FIG. 3A two sub-trees, F and G, are indicated corresponding to overlapping nodes indicated by fragments 6 and 7. Bottom most nodes of FIG. 3A include nodes a, b, c, and d. As is shown, sequence fragment 4 maps to a taxonomic branch that does not overlap with the taxonomic branches of F, G, and G′ in the database at node d. Four associated nodes, a, b, c, and d, are indicated in FIG. 3A.

FIG. 3B illustrates the exemplary fragments mapped to the same database reflected in FIG. 3A wherein nodes are matched to a fixed level of the taxonomy, specifically along the level corresponding to the horizontal dashed line intersecting a′, b′, and d′. As is shown, fragments 2 and 3, which mapped to bottom most nodes b and c in the left-hand panel, are mapped to node b′ in FIG. 3B, which represents a higher level in the taxonomy. This results in three maps, or associated nodes, specifically to nodes a′, b′, and d′ as shown.

The probabilistic scores of nodes a, b, c, and d for the exemplary read on the right hand side of FIG. 3A is such that

p(a)+p(b)+p(c)+p(d)=1.

For example, determining probabilistic scores for the bottom most level in a taxonomy for a set of seven fragments, such as in FIG. 3A, a value sᵢ can be determined. In some embodiments of the invention, sᵢ represents values associated with each fragments, such as size, wherein i=1, 2, 3 . . . n, wherein n is the number of fragments. In some embodiments of the invention, sᵢ is weighted from a distribution. Then, for example, the probabilities associated with nodes a, b, c, and d, can be determined as follows, wherein F and G represent the exemplary taxonomies of the taxonomic tree depicted in FIG. 3A:

$$α = {\sum }_{i = 1}^{7} {s}_{i} , \\ β = α - {s}_{4} , \\ p ( a ) = p ( G ) \times p ( a \vert G ) = \frac{β}{α} \times \frac{{s}_{1} + {s}_{5}}{{s}_{1} + {s}_{2} + {s}_{3} + {s}_{5} + {s}_{6}} , \\ p ( b ) = p ( G ) \times p ( F \vert G ) \times p ( b \vert F ) = \frac{β}{α} \times \frac{{s}_{2} + {s}_{3} + {s}_{6}}{{s}_{1} + {g}_{2} + {s}_{3} + {s}_{5} + {s}_{6}} \times \frac{{s}_{2}}{{s}_{2} + {s}_{3}} , \\ p ( c ) = p ( G ) \times p ( F \vert G ) \times p ( c \vert F ) = \frac{β}{α} \times \frac{{s}_{2} + {s}_{3} + {s}_{6}}{{s}_{1} + {s}_{2} + {s}_{3} + {s}_{5} + {s}_{6}} \times \frac{{s}_{3}}{{s}_{2} + {s}_{3}} , \\ p ( d ) = \frac{α - β}{α} .$$

Of note, p(a)+p(b)+p(c)=β/α; thus p(a)+p(b)+p(c)+p(d)=1.

Determining probabilities for each node of a specified taxonomic level, for instance nodes a′, b′, and d′ as shown in FIG. 3B, can be accomplished as follows, wherein G′ represents the exemplary taxonomy of the taxonomic tree in FIG. 3B:

$$p ( {a}^{′} ) = p ( {G}^{′} ) \times p ( {a}^{′} \vert {G}^{′} ) = \frac{β}{α} \times \frac{{s}_{1} + {s}_{5}}{{s}_{1} + {s}_{2} + {s}_{3} + {s}_{5} + {s}_{6}} , \\ p ( {b}^{′} ) = p ( {G}^{′} ) \times \times p ( {b}^{′} \vert {G}^{′} ) = \frac{β}{α} \times \frac{{s}_{2} + {s}_{3} + {s}_{6}}{{s}_{1} + {s}_{2} + {s}_{3} + {s}_{5} + {s}_{6}} , \\ p ( {d}^{′} ) = \frac{α - β}{α} .$$

Of note, p(a′)+p(b′)=β/α; thus p(a′)+p(b′)+p(d′)=1.

FIG. 3C represents the same exemplary data depicted in FIG. 3A without the node previously marked by sequence 6. As is shown, the only subtree in this instance is subtree G, resulting in s₆=0. The resultant probabilities can be determined as follows:

$$α = {s}_{1} + {s}_{2} + {s}_{3} + {s}_{4} + {s}_{5} + {s}_{7}$$

$$β = α - {s}_{4} , \\ p ( a ) = p ( G ) \times p ( a \vert G ) = \frac{β}{α} \times \frac{{s}_{1} + {s}_{5}}{{s}_{1} + {s}_{2} + {s}_{3} + {s}_{5}} , \\ p ( b ) = p ( G ) \times p ( b \vert G ) = \frac{β}{α} \times \frac{{s}_{2}}{{s}_{1} + {s}_{2} + {s}_{3} + {s}_{5}} , \\ p ( c ) = p ( G ) \times p ( c \vert G ) = \frac{β}{α} \times \frac{{s}_{3}}{{s}_{1} + {s}_{2} + {s}_{3} + {s}_{5}} , \\ p ( d ) = \frac{α - β}{α} .$$

In some embodiments of the invention, a final probability score can be generated for each identification. For example, a species or substrain of a species can be identified as being present in a metagenomic sample with an associated level of confidence, wherein the confidence is based at least in part upon the probabilistic score for one or more underlying associated nodes.

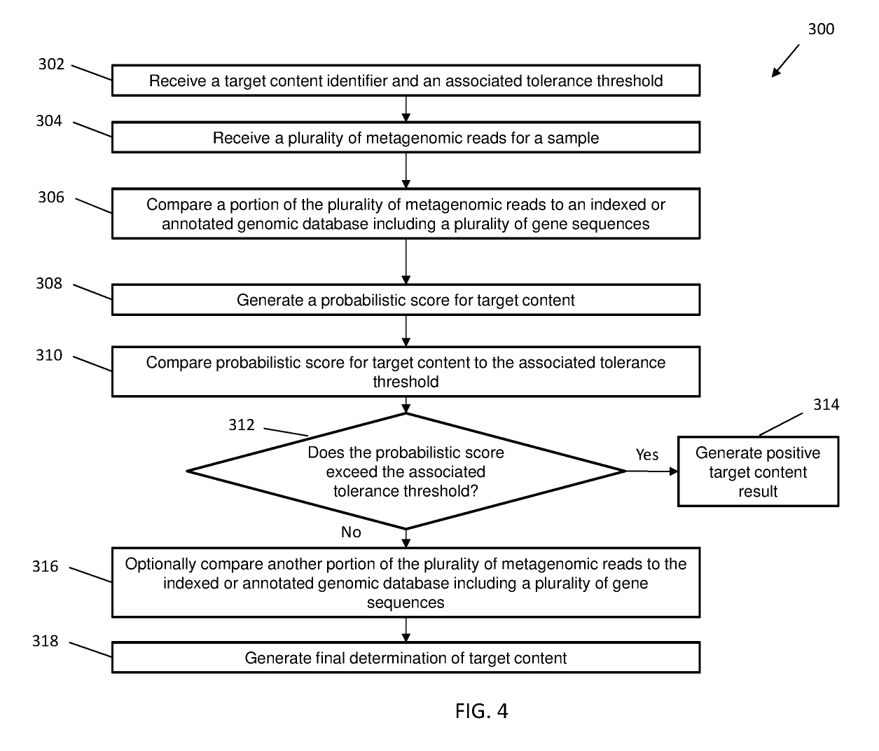

FIG. 4 is a flow diagram illustrating an exemplary method 300 according to one or more embodiments of the present invention. The method 300 includes, as shown at block 302, receiving a target content identifier and an associated tolerance threshold. The method 300 also includes, as shown at block 304, receiving a plurality of metagenomic reads for a sample. The method 300 also includes, as shown at block 306, comparing a portion of the plurality of metagenomic reads to an indexed or annotated genomic database including a plurality of gene sequences.

The method 300 also includes, as shown at block 308 generating a probabilistic score for the target content.

The method 300 also includes, as shown at block 310, comparing the probabilistic score for the target content to the associated tolerance threshold.

The method 300 also includes, as shown at decision block 312, determining whether the probabilistic score exceeds the associated tolerance threshold. If the score exceeds the threshold, the method 300 can proceed to block 314 and generate a positive target content result. For example, the method can indicate that an unacceptable level of salmonella is present in a food sample. If the score does not exceed the threshold, the method 300 can optionally proceed to block 316 and compares another portion of the plurality of metagenomic reads to the indexed or annotated genomic database including a plurality of gene sequences. The method 300 also includes, as shown at block 318, generating a final determination of target content. For example, a final determination of target content can include an indication that target content, such as undesirable content, is absent or that the target content could be present according to a certain probabilistic score.

The target content identifier can be an identification of a taxonomy, nucleic acid sequence, and/or set of nucleic acid sequences associated with content sought to be avoided in a given sample. The target content can be any content that can be identified by a nucleic acid sequence and that is sought to be excluded from a sample or included in a sample. For example, in food applications, undesirable content such as bacteria or other pathogens, such as salmonella, can be target content. In some embodiments of the invention, target content can include desirable content, such as probiotic or other commensal microbes.

The associated tolerance threshold can be based at least in part upon a number of exact matches to a reference database, a percentage of the total sample DNA and/or RNA that matches a database, or a minimum number of reads that matches a given sequence portion, such as a minimum number of reads covering an antibiotic resistance-related mutation. For example, an associated tolerance can be 1% of total sample nucleic acid, at least 100 matching sequencing reads, or at least 10 reads covering a relevant genetic mutation.

In some embodiments of the invention, the method can iteratively or sequentially compare metagenomic reads to a genomic database until a desired stopping point is reached. A desired stopping point can include, for example, a positive target content result, a desired level of accuracy for the analysis, for example, upon achieving 100 million sequencing reads, or a positive identification of a specific organism.

Some embodiments of the invention include building a metagenomic profile based upon the plurality of metagenomic reads. A metagenomic profile can include, for instance, species level abundance. Some embodiments include comparing the metagenomic profile to a pre-determined baseline profile. Some embodiments of the invention include identifying unexpected community variations based at least in part upon the comparison, for instance unexpected community variations above a threshold. In some embodiments of the invention, a probabilistic score is generated for each associated node per set of metagenomic reads.

In some embodiments of the invention, a method includes performing a culture-based or rapid PCR test to confirm a determination of undesired content or a positive target content result.

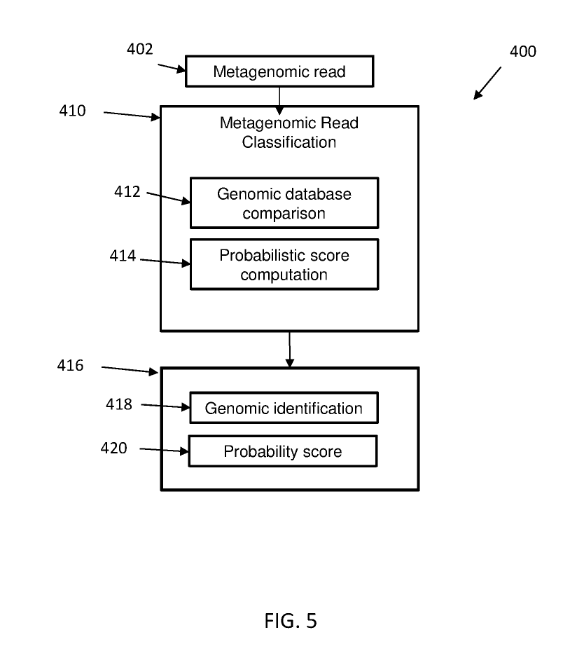

FIG. 5 illustrates an exemplary system 400 for identifying a metagenomic read according to one or more embodiments of the present invention. The system 400 can include an input 402 containing one or more metagenomic reads of a sample. The system 400 can also include a metagenomic read classification engine 410, which can include a genomic database comparison hub 412 and a probabilistic score computation hub 414. The system 400 can also include an output display 416 including, for instance, a genomic identification 418 and a probability score 420 for the genomic identification.

In accordance with embodiments of the invention, any genomic database including known characterizations of gene sequences can be used. In some embodiments of the invention, a plurality of genomic databases are used. Genomic databases that can be used in embodiments of the invention are known, and include, for instance, existing databases such as Silva, Greengenes, NCBI, Ensembl reference genome databases and the like.

Embodiments of the invention can provide rapid identification of target agents, such as viruses or bacteria, for human health and safety issues. For instance, in a hospital setting, visitor saliva can be tested for the presence of target agents prior to entry into areas having immune compromised patients.

### EXAMPLES

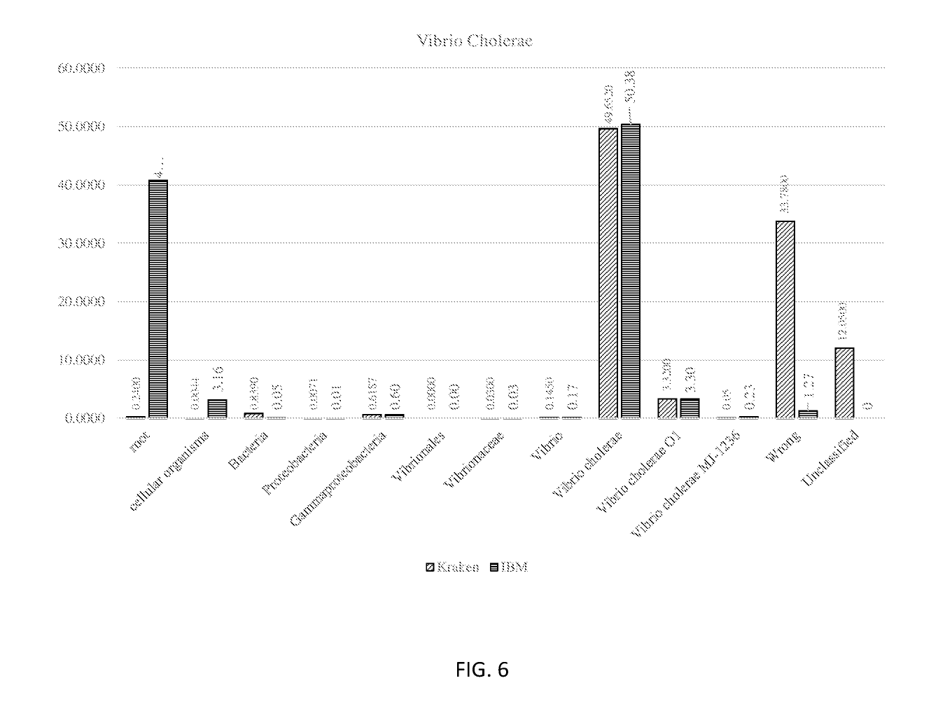

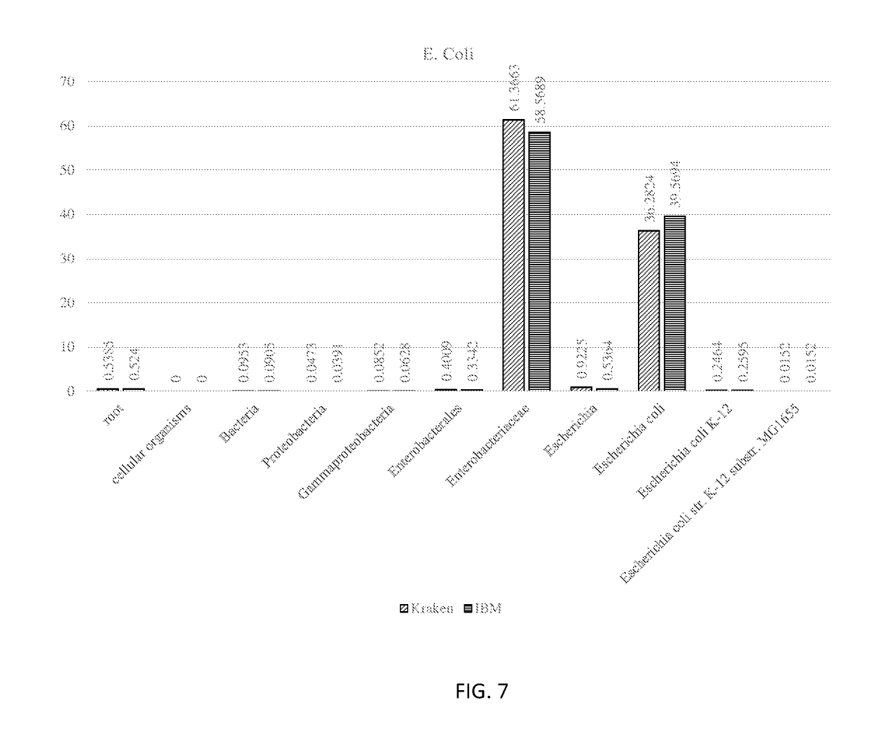

Probabilistic scoring according to embodiments of the invention was compared to a k-mer metagenomic characterization method (“Kraken”) reported in Wood and Salzberg, Genome Biology 2014, 15:R46. Two datasets including two million reads of a sample were applied to a genomic database. The first dataset included V. Cholerae substrain MJ-1236, with results depicted in FIG. 6. The second dataset included E. Coli substrain MG1655, with results depicted in FIG. 7. The approaches assign one read at a time, therefore the input could include reads from a mixture of the two species and achieve identical accuracy. FIGS. 6 and 7 illustrate the percentage of unique reads mapped to specific nodes of the taxonomies T.

As is shown in FIG. 6, embodiments of the invention resulted in a lower number of misclassified (incorrect) identifications relative to Kraken and generated four times the number of reads to the specific substrain, as well as more reads assigned at the species level.

As is shown in FIG. 7, embodiments of the invention assigned 10% more reads to the specific species, E. Coli, than the Kraken method.

Embodiments of the invention can be used to rapidly identify target content in health, and environmental applications. For example, embodiments a positive identification of target content above a probabilistic threshold can lead to destruction of a contaminated product before distribution to the public, to recalling a contaminated product before human consumption, or to quarantining a particular location after identification of environmental hazards.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for identifying content in a metagenomic sample, the method comprising:
receiving, by a processor, a plurality of metagenomic reads for a sample;
comparing, by the processor, a portion of the plurality of metagenomic reads to a genomic database and identifying a plurality of associated nodes, wherein the genomic database comprises a plurality of gene sequences having known taxonomies, and wherein each associated node comprises a match between a metagenomic read and a portion of the known taxonomy;
generating, by the processor, a probabilistic score for each of the associated nodes; and
generating an output comprising a plurality of identifications and a final probability score for each of the identifications based at least in part upon the probabilistic score for each of the associated nodes.

2. The computer-implemented method of claim 1 further comprising
receiving, by the processor, a target content identifier; and
generating, by the processor, a probabilistic score for target content based at least in part upon the comparison of the plurality of metagenomic reads to the genomic database.

3. The computer-implemented method of claim 3 further comprising
receiving, by the processor, an associated tolerance threshold for the target content identifier;
comparing, by the processor, the probabilistic score for target content to the associated tolerance threshold; and
generating, by the processor, a positive target content result based at least in part upon a determination that the probabilistic score exceeds the associated tolerance threshold.

4. The computer-implemented method of claim 1 further comprising
comparing, by the processor, a second portion of the plurality of metagenomic reads to the genomic database;
identifying, by the processor, a second plurality of associated nodes; and
generating, by the processor, a supplemental probabilistic score for each of the second plurality associated nodes per metagenomic read.

5. The computer-implemented method of claim 1, wherein the plurality of metagenomic reads is derived from an environmental sample, a food sample, or a human tissue or fluid sample.

6. The computer-implemented method of claim 1 further comprising
building, by the processor, a metagenomic profile based at least in part upon the plurality of metagenomic reads;
comparing, by the processor, the metagenomic profile to a pre-determined baseline profile; and
identifying, by the processor, an unexpected community variation based at least in part upon the comparison.

7. The computer-implemented method of claim 1 further comprises repeating, by the processor, the comparison of the plurality of metagenomic reads to the genomic database until a threshold accuracy is achieved.

8. A computer program product for identifying content in a metagenomic sample, the computer program product comprising:
a computer readable storage medium readable by a processing circuit and storing program instructions for execution by the processing circuit for performing a method comprising:
receiving a plurality of metagenomic reads for a sample;
comparing a portion of the plurality of metagenomic reads to a genomic database and identifying a plurality of associated nodes, wherein the genomic database comprises a plurality of gene sequences having known taxonomies;
generating a probabilistic score for each of the associated nodes per metagenomic read; and
generating an output comprising a plurality of identifications and a final probability score for each of the identifications based at least in part upon the probabilistic score for each of the associated nodes per metagenomic read.

9. The computer program product of claim 8, wherein the method further comprises
receiving a target content identifier; and
generating a probabilistic score for target content based at least in part upon the comparison of the plurality of metagenomic reads to the genomic database.

10. The computer program product of claim 9, wherein the method further comprises
receiving an associated tolerance threshold for the target content identifier;
comparing the probabilistic score for target content to the associated tolerance threshold; and
generating a positive target content result based at least in part upon a determination that the probabilistic score exceeds the associated tolerance threshold.

11. The computer program product of claim 8, wherein the method further comprises
comparing a second portion of the plurality of metagenomic reads to the genomic database;
identifying a second plurality of associated nodes; and
generating a supplemental probabilistic score for each of the second plurality associated nodes per metagenomic read.

12. The computer program product of claim 8, wherein the plurality of metagenomic reads is derived from an environmental sample, a food sample, or a human tissue or fluid sample.

13. The computer program product of claim 8, wherein the method further comprises building a metagenomic profile based at least in part upon the plurality of metagenomic reads;
comparing the metagenomic profile to a pre-determined baseline profile; and
identifying an unexpected community variation based at least in part upon the comparison.

14. The computer program product of claim 8, wherein the method further comprises repeating the comparison of the plurality of metagenomic reads to the genomic database until a threshold accuracy is achieved.

15. A processing system for identifying content in a metagenomic sample, the system comprising:
a processor in communication with one or more types of memory, the processor configured to perform a method comprising:
receiving a plurality of metagenomic reads for a sample;
comparing a portion of the plurality of metagenomic reads to a genomic database and identifying a plurality of associated nodes, wherein the genomic database comprises a plurality of gene sequences having known taxonomies;
generating a probabilistic score for each of the associated nodes per metagenomic read; and
generating an output comprising a plurality of identifications and a final probability score for each of the identifications based at least in part upon the probabilistic score for each of the associated nodes per metagenomic read.

16. The processing system of claim 15, wherein the wherein the method further comprises
receiving a target content identifier; and
generating a probabilistic score for target content based at least in part upon the comparison of the plurality of metagenomic reads to the genomic database.

17. The processing system of claim 16, wherein the method further comprises
receiving an associated tolerance threshold for the target content identifier;
comparing the probabilistic score for target content to the associated tolerance threshold; and
generating a positive target content result based at least in part upon a determination that the probabilistic score exceeds the associated tolerance threshold.

18. The processing system of claim 15, wherein the method further comprises
comparing a second portion of the plurality of metagenomic reads to the genomic database;
identifying a second plurality of associated nodes; and
generating a supplemental probabilistic score for each of the second plurality associated nodes per metagenomic read.

19. The processing system of claim 15, wherein the plurality of metagenomic reads is derived from an environmental sample, a food sample, or a human tissue or fluid sample.

20. The processing system of claim 15, wherein the method further comprises building a metagenomic profile based at least in part upon the plurality of metagenomic reads;
comparing the metagenomic profile to a pre-determined baseline profile; and
identifying an unexpected community variation based at least in part upon the comparison.
